(19) United States
(12) Patent Application Publication
(10) Pub. No.: US 2006/0008517 A1
(43) Pub. Date: Jan. 12, 2006

(54) Treatment of age-related memory impairment

(76) Inventors: Marina A. Lynch, Rathgar (IE); Arkady Mandel, North York (CA); Anthony E. Bolton, Santry (IE)

Correspondence Address: FOLEY & LARDNER LLP, 1530 PAGE MILL ROAD, PALO ALTO, CA, 94304, US

(21) Appl. No.: 10/888,343

(22) Filed: Jul. 9, 2004

(51) Int. Cl. A61K 9/127

(52) U.S. Cl. 424/450

(57) ABSTRACT

Symptoms, including biochemical correlates, of age-related memory loss (ARML) in a mammal are beneficially affected by administering to the mammal small doses of bodies, such as liposomes, of a size resembling that of mammalian cells, the bodies having phosphate glycerol head groups presented exteriorly on their surfaces. Preferred are liposomes comprised of 50-100% phosphatidylglycerol, with the phosphoglycerol headgroups thereof exteriorly presented.

### BACKGROUND OF THE INVENTION

1. Field of Invention

This Invention relates to medical treatments and compositions useful in treatments for improving neurological function, especially the neurological functioning of aged mammals.

2. Background of the Invention

It is well known that brain function generally deteriorates as individuals age. Specifically, declines in memory and cognitive abilities occur with age in virtually all mammalian species. Such general deterioration of cerebral function is distinct from that associated with age-related dementias, such as Alzheimer's disease and Parkinson's disease, which involve neurological deterioration and attendant pathophysiology of a clinically defined type. In contrast, age-related memory impairment is characterized by a gradual loss of memory and cognitive function.

Age related cognitive decline and memory impairment, hereinafter referred to as “age related memory loss” (ARML) is not a form of dementia, nor a form of impaired motor function. While dementia involves a broad loss of cognitive abilities, ARML is primarily a deficit of declarative memory, with variable components of impairment of cognitive (thinking, reasoning, learning) function, which is, to a greater or lesser extent, considered to be a natural consequence of the aging process.

Changes in brain performance initially occur in the memory, as an individual ages. The working-memory capacity becomes more limited, as the frontal cortex of the brain Is less able to sustain a sufficient working memory. Further, more time is needed to learn new information. As a result of these combined deficits in memory and cognition, the subject loses his or her ability to keep several items of information in the working memory at the same time, when faced with delay or distraction.

ARML thus relates to a progressive deterioration of neurological functioning, that does not rise to the level of dementias, such as is seen in Alzheimer's disease and Parkinson's disease, nor to the level of conditions of mental retardation such as Down's syndrome.

The neuropathology of ARML may include decreased brain weight, gyral atrophy, ventricular dilation, and selective loss of neurons within different brain regions, as well as low levels of plaques or neurofibrillatory tangles; however, these pathological findings are in no way as pronounced as the neuronal loss, plaques and neurofibrillatory tangles that are the hallmarks of dementias such as Alzheimer's disease. Furthermore, ARML subjects can be distinguished from dementia patients by virtue of the fact that ARML subjects score within a normal range on standardized diagnostic tests for dementias, such as the Diagnostic and Statistical Manual of Mental Disorders: 4th Edition of the American Psychiatric Association (DSM-IV, 1994); this standardized testing paradigm provides separate diagnostic criteria for the condition termed “Age-Related Cognitive Decline (ARCD),” which is synonymous with the term ARML, as described below.

Scientific study and analysis of subtle changes in memory as occur in ARML have been limited in the past by lack of objective measurements in humans and lack of dependable animal models. Thus, human measurements have relied in large part on anecdotal evidence from the patient or the patient's family. Similarly, animal measurements of memory were carried out using crude, largely behavioral indices, such as performance in animal mazes. In recent years, however, scientists have developed a number of objective measures and biochemical correlates of memory function in animal models. While it has been known for some time that the hippocampal region of the brain plays a significant role in learning and memory, recently, brain levels of certain cytokines and other biological markers have been correlated with age and/or memory function. For example, Increased concentrations in the hippocampus of the pro-inflammatory cytokine interleukin 1β (IL-Iβ) are accompanied by an impairment of hippocampal-dependent learning and memory (Shaw K. N. et al. (2001) Behav Brain Res 124: 47-54). Elderly rats show age-related changes in hippocampal function attributable to increased IL1-β concentration, and deficits in long-term potentiation (Murray C and Lynch M A (1998) J Neurosci 18:2974-2981).

Similarly, scientists have developed electrophysiological measurements in hippocampus that provide ways of assessing synaptic function in test animals. For example, long-term potentiation (L TP) is a form of synaptic plasticity that can be measured experimentally in the hippocampal formation of test animals, such as rats. LTP is now generally accepted as a biological substrate for learning and memory (see Bliss et al., (1990) Nature 361: 31-39). Reduction in LTP indicates a reduction in synaptic function and a concomitant reduction in memory and cognitive function. Electrophysiological recording of LTP in the rat hippocampus therefore provides a means of assessing synaptic function and consequently cognitive function and memory and cognitive function. Thus, there are now ways of testing treatment modalities for ability to effect these more subtle indicators of memory and cognitive function. In addition to the inverse correlation of IL-1β and LTP, impaired LTP is also associated with increases in the concentration of interferon-gamma (IFN-γ) in the hippocampus.

While considerable efforts have been expended to find treatments or cures for Alzheimer's disease and other forms of dementia, treatment of general memory and cognitive impairment associated with old age has been left largely to behavioral forms of therapy. Therefore, therapies that would reduce or lessen the effects of aging on neurological (synaptic) function would likely be useful to the aging population.

### SUMMARY OF THE INVENTION

The present invention provides methods for reducing the progression of age related loss of neurological function in a mammal subject. Specifically, the present invention provides for the deceleration, cessation and/or reversal, in some instances, of one or more symptoms of age-related memory and/or cognition impairment, as herein defined.

Underlying the present invention is the observation that the hippocampal concentrations of certain pro-inflammatory cytokines have been shown to change with age in mammals. Without ascribing to any particular theory, it is hypothesized that improvement of memory and cognitive function may be mediated by reversing or attenuating such changes.

Studies carried out in support of the present invention, as described herein, show that administration of phosphatidylglycerol-carrying bodies to aged rats attenuates or reverses certain biochemical and electrophysiological changes associated with the aged brain. Thus, in studies carried out in support of the present invention, administration of compositions of the invention is shown to reduce levels of certain biochemical markers (IFN-γ, IL-1β, pJNK) that are normally elevated and to increase the levels of other markers (pERK) that are normally lowered in the hippocampi of aged rats. Concomitant with such effects, measurement of synaptic function in a standard animal model of brain function, namely long term potentiation (LTP) in the rat hippocampus, reveals improvement of function in the hippocampi of aged rats, following administration of compositions of the invention. The invention thus shows the potential for halting and even reversing age related memory loss (ARML) in mammals, such as humans.

In accordance with the present invention, an appropriate dosage of three-dimensional synthetic or semi-synthetic bodies is administered to an aging mammal showing or likely to show symptoms of ARML. Such bodies have shapes and dimensions ranging from those resembling mammalian cells to shapes and dimensions approximating to apoptotic bodies produced by apoptosis of mammalian cells, and having phosphate-glycerol molecules on the surface thereof.

According to one embodiment of the invention, PG-carrying bodies may be administered as liposomes comprising 50-100% by weight of phosphatidylglycerol on their surfaces. Preferably, PG-carrying bodies have diameters from about 50 nanometers to about 1000 nanometers (0.05-1 micron).

According to another feature, PG-carrying bodies are administered in a unit dosage amount of from about 500 to about 5×10¹² bodies per unit dosage. Such administration may be by any of a number of routes, including, without limitation, intramuscular administration.

PG-carrying bodies, as described above and herein, may also be used in the preparation of medicaments for reducing, treating or preventing age-related memory loss in mammalian subjects.

These and other objects and features of the invention will become more fully apparent when the following detailed description of the invention is read in conjunction with the accompanying drawings.

All publications cited herein are herein incorporated by reference in their entirety to the same extent as if each individual publication was specifically and individually incorporated by reference in its entirety.

### BRIEF DESCRIPTION OF THE DRAWINGS

FIG. 1 is a graph of the epsp slope against time, for young animals, treated and untreated according to the invention, and aged animals, untreated and treated according to the invention, demonstrating improvement in long term potentiation (LTP) in the hippocampus of aged rats, resulting from the preferred embodiment of the invention.

FIG. 2 is a graphical presentation of the FIG. 1 data in the form of a percentage change in epsp slope, for young and aged animals, controls and treated according to the preferred embodiment of the invention.

FIG. 3 is a bar graph showing interferon-gamma (IFN-γ) levels in the hippocampi of young and aged rats treated with saline (control; open bars) or PG liposomes (PG; cross-hatched bars).

FIG. 4 is a bar graph showing interleukin I-beta (1L-1β) measurements in the hippocampi of young and aged rats treated with saline (control; open bars) or PG liposomes (PG; cross-hatched bars).

FIG. 5 is a bar graph showing c-Jun-N-terminal protein kinase (p-JNK) measurements in the hippocampi of young and aged rats treated with saline (control; open bars) or PG liposomes (PG; cross-hatched bars).

FIG. 6 is a bar graph showing pro-survival extracellular regulated kinase (pERK, an enzyme associated with cell survival) phosphorylation activity measurement in the hippocampi of young and aged rats treated with saline (control; open bars) or PG liposomes (PG; cross-hatched bars).

### DETAILED DESCRIPTION OF THE PREFERRED EMBODIMENTS

I. Definitions

This section sets forth certain defined terms; other terms used herein are defined In context and/or have the meanings generally attributable to them in standard usage by those skilled in the art.

The term “age-related memory loss” (abbreviated “ARML”), refers to any of a continuum of conditions characterized by a deterioration of neurological functioning that does not rise to the level of a dementia, as further defined herein and/or as defined by the Diagnostic and Statistical Manual of Mental Disorders: 4th Edition of the American Psychiatric Association (DSM-IV, 1994). This term specifically excludes age-related dementias such as Alzheimer's disease and Parkinson's disease, and conditions of mental retardation such as Down's syndrome. ARML is characterized by objective loss of memory in an older subject compared to his or her younger years, but cognitive test performance that is within normal limits for the subject's age. ARML subjects score within a normal range on standardized diagnostic tests for dementias, as set forth by the DSM-IV. Moreover, the DSM-IV provides separate diagnostic criteria for a condition termed “Age-Related Cognitive Decline (ARCD)”. In the context of the present invention, ARCD, as well as the terms “Age-Associated Memory Impairment (AAMI)” and “Age-Consistent Memory Decline (ACMD)” are understood to be synonymous with the term ARML. Age-related memory loss may include decreased brain weight, gyral atrophy, ventricular dilation, and selective loss of neurons within different brain regions. For purposes of the preferred embodiments of the present invention, more progressive forms of memory loss are also included under the definition of age-related memory disorder. Thus persons having greater than age-normal memory loss and cognitive impairment, yet scoring below the diagnostic threshold for frank dementia, may be referred to as having a mild neurocognitive disorder, mild cognitive impairment, late-life forgetfulness, benign senescent forgetfulness, incipient dementia, provisional dementia, and the like. Such subjects may be slightly more susceptible to developing frank dementia in later life.

The term “biocompatible” refers to substances that, in the amount employed, are either non-toxic or have acceptable toxicity profiles such that their use in vivo is acceptable.

The term “cognitive dysfunction” or “cognitive impairment” refers to difficulties in thinking, reasoning or problem-solving.

The term “dementia” refers to any of a number of chronic or persistent mental disorders marked by memory failures, personality changes and impaired reasoning (Concise Oxford Dictionary, 10th edition; National Institute on Aging, www.niapublications.org/engagepages/forgetfulness.asp). It may result from many illnesses, including Alzheimer's disease, AIDS, chronic alcoholism, vitamin B-12 deficiency, CO poisoning, among others. A common type of dementia in older people is “multi-infarct” dementia, which is also referred to as “vascular dementia.” This form of dementia is the result of a series of small strokes or transient ischemic attacks, which result in neuronal death. The symptoms and seriousness of this form of dementia is highly dependent upon the part(s) of the brain deprived of blood flow during the attacks. A diagnosis of dementia can be made based on DSM-IV criteria.

The terms “liposomes” and “lipid vesicles” refer to sealed membrane sacs, having diameters in the micron or sub-micron range, the walls of which consist of layers, typically bilayers, of suitable, membrane-forming amphiphiles. They normally contain an aqueous medium.

The term “pharmaceutically acceptable” has a meaning that is similar to the meaning of the term “biocompatible.” As used in relation to “pharmaceutically acceptable bodies” herein, it refers to bodies of the invention comprised of one or more materials which are suitable for administration to a mammal, preferably a human, in viva, according to the method of administration specified (e.g., intramuscular, intravenous, subcutaneous, topical, oral, and the like).

The term “phosphate choline” refers to the group —O—P(═O)(OH)—O—CH2—CH2—N+(CH3)3, which can attached to lipids to form “phosphatidylcholine” (PC) as shown in the following structure: 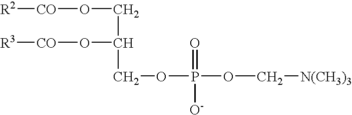
and salts thereof, wherein R2 and R3 are independently selected from Cl-C24 hydrocarbon chains, saturated or unsaturated, straight chain or containing a limited amount of branching wherein at least one chain has from 10-24 carbon atoms.

The term “phosphate-glycerol-carrying bodies” refers to biocompatible, pharmaceutically-acceptable, three-dimensional bodies having on their surfaces phosphate-glycerol groups or groups that can be converted to phosphate-glycerol groups, as described herein.

A “phosphate-glycerol group” is a group having the general structure: O—P(═O)(OH)—O—CH₂CH(OH)CH₂OH, and derivatives thereof, including, but not limited to groups in which the negatively charged oxygen of the phosphate group of the phosphate-glycerol group is converted to a phosphate ester group (e.g., L-OP(O)(OR′)(OR″), where L is the remainder of the phosphate-glycerol group, R′ is-CH₂CH(OH)CH₂OH and R″ is alkyl of from 1 to 4 carbon atoms, or a hydroxyl substituted alkyl of from 2 to 4 carbon atoms, and 1 to 3 hydroxyl groups provided that R″ is more readily hydrolyzed in vivo than the R′ group; to a diphosphate group including diphosphate esters (e.g., L-OP(O)(OR′)OP(O)(OR″)₂ wherein L and R′ are as defined above and each R″ is independently hydrogen, alkyl of from 1 to 4 carbon atoms, or a hydroxyl substituted alkyl of from 2 to 4 carbon atoms and 1 to 3 hydroxyl groups, provided that the second phosphate [—P(O)(OR″)₂] is more readily hydrolyzed in vivo than the R′ group; or to a triphosphate group including triphosphate esters (e.g., L-OP(O)(OR′)OP(O)(OR″)OP(O)(OR″)₂ wherein L and R′ are defined as above and each R″ is independently hydrogen, alkyl of from 1 to 4 carbon atoms, or a hydroxyl substituted alkyl of from 2 to 4 carbon atoms and 1 to 3 hydroxyl groups provided that the second and third phosphate groups are more readily hydrolyzed in vivo than the R′ group; and the like. Such synthetically altered phosphate-glycerol groups are capable of expressing phosphate-glycerol in vivo and, accordingly, such altered groups are phosphate-glycerol convertible groups within the scope of the invention. A specific example of a phosphate-glycerol group is the compound phosphatidylglycerol (PG), further defined herein.

“Phosphatidylglycerol” is also abbreviated herein as “PG.” This term is intended to cover phospholipids carrying a phosphate-glycerol group with a wide range of at least one fatty acid chain provided that the resulting PG entity can participate as a structural component of a liposome. Chemically, PG has a phosphate-glycerol group and a pair of similar, but different fatty acid side chains. Preferably, such PG compounds can be represented by the Formula I: 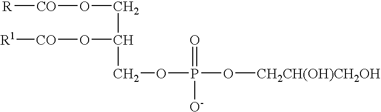
where R and R¹ are independently selected from C₁-C₂₄ hydrocarbon chains, saturated or unsaturated, straight chain or containing a limited amount of branching wherein at least one chain has from 10 to 24 carbon atoms. R and R¹ can be varied to include two or one lipid chain(s), which can be the same or different, provided they fulfill the structural function. As mentioned above, the fatty acid side chains may be from about 10 to about 24 carbon atoms in length, saturated, mono-unsaturated or polyunsaturated, straight-chain or with a limited amount of branching. Laurate (C12), myristate (C14, palmitate (C16), stearate (C18), arachidate (C20), behenate (C22) and lignocerate (C24) are examples of useful saturated fatty acid side chains for the PG for use in the present invention. Palmitoleate (C15), oleate (C18) are examples of suitable mono-unsaturated fatty acid side chains. Linoleate (C18), linolenate (C18) and arachidonate (C20) are examples of suitable polyunsaturated fatty acid side chains for use in PG in the compositions of the present invention. Phospholipids with a single such fatty acid side chain, also useful in the present invention, are known as lysophospholipids.

The term PG also includes dimeric forms of PG, namely cardiolipin, but other dimers of Formula I are also suitable. Preferably, such dimers are not synthetically cross-linked with a synthetic cross-linking agent, such as maleimide but rather are cross-linked by removal of a glycerol unit as described by Lehninger, Biochemistry and depicted in the reaction below: 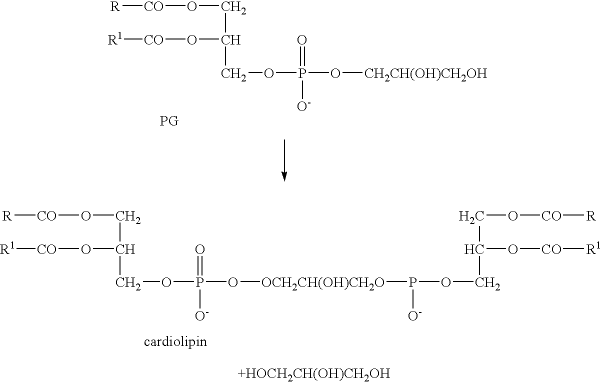
Purified forms of phosphatidylglycerol are commercially available, for example, from Sigma-Aldrich (St. Louis, Mo.). Alternatively, PG can be produced, for example, by treating the naturally occurring dimeric form of phosphatidylglycerol, cardiolipin, with phospholipase D. It can also be prepared by enzymatic synthesis from phosphatidyl choline using phospholipase D (see, for example, U.S. Pat. No. 5,188,951 (Tremblay et al., incorporated herein by reference).

“PG-carrying bodies” are three-dimensional bodies, as described above, that have surface PG molecules. By way of example, PG can form the membrane of a liposome, either as the sole constituent of the membrane or as a major or minor component thereof, with other phospholipids and/or membrane forming materials.

The term “phosphatidylserine” or “PS” is intended to cover phosphatidyl serine and analogs/derivatives thereof.

The term “symptoms associated with age-related memory loss” includes one or more of a variety of attributes of age-related memory loss, including, but not limited to alterations in biochemical markers associated with the aging brain, such as IL-1β, IFN-γ, p-JNK, p-ERK, reduction in synaptic activity or function, such as synaptic plasticity, evidenced by reduction in long term potentiation (LTP), diminution of memory, reduction of cognition.

The term “synaptic function” refers to electrophysiological correlates of brain activity including synaptic plasticity, measured by long term potentiation (LTP), as well as electroencephalogram activity.

In the context of the present invention, “three-dimensional bodies” refer to biocompatible synthetic or semi-synthetic entities, including but not limited to liposomes, solid beads, hollow beads, filled beads, particles, granules and microspheres of biocompatible materials, natural or synthetic, as commonly used in the pharmaceutical industry. Liposomes may be formed of lipids, including phosphatidylglycerol (PG). Beads may be solid or hollow, or filled with a biocompatible material. Such bodies have shapes that are typically, but not exclusively spheroidal, cylindrical, ellipsoidal, including oblate and prolate spheroidal, serpentine, reniform and the like, and have sizes ranging from 200 nm to 500 μm, preferably measured along the longest axis.

II. Phosphate-Glycerol-Carrying Bodies

This section describes various embodiments of phosphate-glycerol-carrying bodies contemplated by the present invention, including specific embodiments thereof. With the guidance provided herein, persons having requisite skill in the art will readily understand how to make and use phosphate-glycerol-carrying bodies in accordance with the present invention.

In the context of the present invention, phosphate-glycerol-carrying bodies refer to biocompatible, pharmaceutically-acceptable, three-dimensional bodies having on their surfaces phosphate-glycerol groups or groups that can be converted to phosphate-glycerol groups, as described herein.

A. Phosphate-Glycerol Groups

According to a general feature of the invention, phosphate-glycerol groups useful in the present invention have the general structure:
O—P(═O)(OH)—O—CH₂CH(OH)CH₂OH
Such phosphate-glycerol groups include synthetically altered versions of the phosphate-glycerol group shown above, and may include all, part of or a modified version of the original phosphate-glycerol group.

Preferably the fatty acid side chains of the chosen PG will be suitable for formation of liposomes, and incorporation into the lipid membrane(s) forming such liposomes, as described in more detail below.

More generally, without being limited to any particular theory, it is believed that phosphate-glycerol groups according to the present invention are capable of interacting with one or more receptors present in relevant brain tissue, such as the hippocampus. A specific example of a phosphate-glycerol group is the compound phosphatidylglycerol (PG), described above.

PG groups of the present invention, including dimers thereof, are believed to act as ligands, binding to specific sites on a protein or other molecule (“PG receptor”) and, accordingly, PG (or derivatives or dimeric forms thereof) are sometimes referred to herein as a “ligand” or a “binding group.” Such binding is believed to take place through the phosphate-glycerol group —O—P(═O)(OH)—O—CH₂CH(OH)CH₂OH, which is sometimes referred to herein as the ahead group, “active group,” or “binding group,” while the fatty acid side chain(s) are believed to stabilize the group and/or, in the case of liposomal preparations, form the outer lipid layer or layer of the liposome. More generally, again without being limited to any particular theory, it is believed that phosphate-glycerol groups, including PG are capable of interacting with one or more receptors in the brain and that such interactions may provide positive effects on synaptic transmission, and, by extension, memory, as described herein.

B. Formation of Phosphate-Glycerol Carrying Bodies

Phosphate-glycerol carrying bodies are three-dimensional bodies that have surface phosphate-glycerol molecules. This section will describe general and exemplary phosphate-glycerol carrying bodies suitable for use in the present invention.

Generally, phosphate-glycerol carrying bodies of the present invention carry phosphate-glycerol molecules on their exterior surfaces to facilitate in vivo interaction of the binding groups.

Three-dimensional bodies are preferably formed to be of a size or sizes suitable for administration to a living subject, preferably by injection; hence such bodies will preferably be in the range of 20 to 1000 nm (0.02-1 micron), more preferably 20 to 500 nm (0.02-0.5 micron), and still more preferably 20-200 nm in diameter, where the diameter of the body is determined on its longest axis, in the case of non-spherical bodies. Suitable sizes are generally in accordance with blood cell sizes. While bodies of the invention have shapes that are typically, but not exclusively spheroidal, they can alternatively be cylindrical, ellipsoidal, including oblate and prolate spheroidal, serpentine, reniform in shape, or the like.

Suitable forms of bodies for use in the compositions of the present invention include, without limitation, particles, granules, microspheres or beads of biocompatible materials, natural or synthetic, such as polyethylene glycol, polyvinylpyrrolidone, polystyrene, and the like; polysaccharides such as hydroxethyl starch, hydroxyethylcellulose, agarose and the like; as are commonly used in the pharmaceutical industry. Preferably, such materials will have side-chains or moieties suitable for derivatization, so that a phosphate-glycerol group, such as PG, may be attached thereto, preferably by covalent bonding. Bodies of the invention may be solid or hollow, or filled with biocompatible material. They are modified as required so that they carry phosphate-glycerol molecules, such as PG on their surfaces. Methods for attaching phosphate-glycerol in general, and PG in particular, to a variety of substrates are known in the art.

In addition to the various bodies listed above, the liposome is a particularly useful form of body for use in the present invention. Liposomes are microscopic vesicles composed of amphiphilic molecules forming a monolayer or bilayer surrounding a central chamber, which may be fluid-filled. Amphipllilic molecules (also referred to as “amphiphiles”), are molecules that have a polar water-soluble group attached to a water-insoluble (lipophilic) hydrocarbon chain, such that a matrix of such molecules will typically form defined polar and apolar regions. Amphiphiles include naturally occurring lipids such as PG, phosphatidylserine, phosphatidylethanolamine, phosphatidylinositol, phosphatidylcholine, cholesterol, cardiolipin, ceramides and sphingomyelin, used alone or in admixture with one another. They can also be synthetic compounds such as polyoxyethylene alkyl ethers, polyoxyethylene alkyl esters and saccharosediesters.

Preferably, for use in forming liposomes, the amphiphilic molecules will include one or more forms of phospholipids of different headgroups (e.g., phosphatidylglycerol, phosphatidylserine, phosphatidylcholine) and having a variety of fatty acid side chains, as described above, as well as other lipophilic molecules, such as cholesterol, sphingolipids and sterols.

In accordance with the present invention, phosphatidylglycerol (PG) will constitute the major portion or the entire portion of the liposome layer(s) or wall(s), oriented so that the phosphate-glycerol group portion thereof is presented exteriorly, as described above, while the fatty acid side chains form the structural wall. When, as in the present invention, the bilayer includes phospholipids, the resulting membrane is usually referred to as a “phospholipid bilayer,” regardless of the presence of non-phospholipid components therein.

Liposomes of the invention are typically formed from phospholipid bilayers or a plurality of concentric phospholipid bilayers which enclose aqueous phases. In some cases, the walls of the liposomes may be single layered; however, such liposomes (termed “single unilamellar vesicles” or “SUVs”) are generally much smaller (diameters less than about 70 nm) than those formed of bilayers, as described below. Liposomes formed in accordance with the present invention are designed to be biocompatible, biodegradable and non-toxic. Liposomes of this type are used in a number of pharmaceutical preparations currently on the market, typically carrying active drug molecules in their aqueous inner core regions. In the present invention, however, the liposomes are not filled with pharmaceutical preparation. The liposomes are active themselves, not acting as drug carrier.

Preferred PG-carrying liposomes of the present invention are constituted to the extent of 50% -100% by weight of phosphatidyl glycerol, the balance being phosphatidylcholine (PC) or other such biologically acceptable phospholipid(s). More preferred are liposomes constituted by PG to the extent of 65% -90% by weight, most preferably 70% -80% by weight, with the single most preferred embodiment, on the basis of current experimental experience, being PG 75% by weight, the balance being other phospholipids such as PC. Such liposomes are prepared from mixtures of the appropriate amounts of phospholipids as starting materials, by known methods. According to an important feature of the invention, PG-carrying bodies comprise less than 50%, preferably less than 40%, still preferably less than 25% and even still preferably less than 10% phosphatidyl serine.

The present invention contemplates the use, as PG-carrying bodies, not only of those liposomes having PG as a membrane constituent, but also liposomes having non-PG membrane substituents that carry on their external surface molecules of phosphate-glycerol, either as monomers or oligomers (as distinguished from phosphatidylglycerol), e.g., chemically attached by chemical modification of the liposome surface of the body, such as the surface of the liposome, making the phosphate-glycerol groups available for subsequent interaction. Because of the inclusion of phosphate-glycerol on the surface of such molecules, they are included within the definition of PG-carrying bodies.

Liposomes may be prepared by a variety of techniques known in the art, such as those detailed in Szoka et al. (Ann. Rev. Biophys. Bioeng. 9:467 (1980)). Depending on the method used for forming the liposomes, as well as any after-formation processing, liposomes may be formed in a variety of sizes and configurations. Methods of preparing liposomes of the appropriate size are known in the art and do not form part of this invention. Reference may be made to various textbooks and literature articles on the subject, for example, the review article by Yechezkel Barenholz and Daan J. A. Chromeline, and literature cited therein, for example New, R. C. (1990), and Nassander, U. K., et al. (1990), and Barenholz, Y and Lichtenberg, D., Liposomes: preparation, characterization, and preservation. Methods Biochem Anal. 1988, 33:337-462.

Multilamellar vesicles (MLVs) can be formed by simple lipid-film hydration techniques according to methods known in the art. In this procedure, a mixture of liposome-forming lipids is dissolved in a suitable organic solvent. The mixture is evaporated in a vessel to form a thin film on the inner surface of the vessel, to which an aqueous medium is then added. The lipid film hydrates to form MLVs, typically with sizes between about 100-1000 nm (0.1 to 10 microns) in diameter.

A related, reverse evaporation phase (REV) technique can also be used to form unilamellar liposomes in the micron diameter size range. The REV technique involves dissolving the selected lipid components, in an organic solvent, such as diethyl ether, in a glass boiling tube and rapidly injecting an aqueous solution, into the tube, through a small gauge passage, such as a 23-gauge hypodermic needle. The tube is then sealed and sonicated in a bath sonicator. The contents of the tube are alternately evaporated under vacuum and vigorously mixed, to form a final liposomal suspension.

By way of example, but not limitation, Example 1 provides a detailed description of a method of preparing a PG-liposomal preparation for use in the present invention.

The diameters of the PG-carrying liposomes of the preferred embodiment of this invention range from about 20 nm to about 1000 nm, more preferably from about 20 nm to about 500 nm, and most preferably from about 20 nm to about 200 nm. Such preferred diameters will correspond to the diameters of mammalian apoptotic bodies, such as may be apprised from the art.

One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. This method of liposome sizing is used in preparing homogeneous-size REV and MLV compositions. U.S. Pat. Nos. 4,737,323 and 4,927,637, incorporated herein by reference, describe methods for producing a suspension of liposomes having uniform sizes in the range of 0.1-0.4 μm (100-400 nm) using as a starting material liposomes having diameters in the range of 1 μm. Homogenization methods are also useful for down-sizing liposomes to sizes of 100 nm or less (Martin, F. J. (1990) In: Specialized Drug Delivery Systems—Manufacturing and Production Technology, P. Tyle (ad.) Marcel Dekker, New York, pp. 267-316.). Another way to reduce liposomal size is by application of high pressures to the liposomal preparation, as in a French Press.

Liposomes can be prepared to have substantially homogeneous sizes of single, bi-layer vesicles in a selected size range between about 0.07 and 0.2 microns (70-200 nm) in diameter, according to methods known in the art. In particular, liposomes in this size range are readily able to extravasate through blood vessel epithelial cells into surrounding tissues. A further advantage is that they can be sterilized by simple filtration methods known in the art.

Whilst a preferred embodiment of PG-carrying bodies for use in the present invention is liposomes with PG presented on the external surface thereof, it is understood that the PG-carrying body is not limited to a liposomal structure, as mentioned above.

III. Dosages and Modes of Administration

The phosphate-glycerol-carrying bodies of the invention may be administered to the patient by any suitable route of administration, including oral, nasal, topical, rectal, intravenous, subcutaneous and intramuscularly. At present, intramuscular administration is preferred, especially in conjunction with PG-liposomes.

The PG-carrying bodies may be suspended in a pharmaceutically acceptable carrier, such as physiological sterile saline, sterile water, pyrogen-free water, isotonic saline, and phosphate buffer solutions, as well as other non-toxic compatible substances used in pharmaceutical formulations. Preferably, PG-carrying bodies are constituted into a liquid suspension in a biocompatible liquid such as physiological saline and administered to the patient in any appropriate route which introduces it to the immune system, such as intra-arterially, intravenously, intra-arterially or most preferably intramuscularly or subcutaneously.

A preferred manner of administering the PG-carrying bodies to the patient is a course of injections, administered daily, several times per week, weekly or monthly to the patient, over a period ranging from a week to several months. The frequency and duration of the course of the administration is likely to vary from patient to patient, and according to the condition being treated, its severity, and whether the treatment is intended as prophylactic, therapeutic or curative. Its design and optimization is well within the skill of the attending physician. In studies carried out in support of the present invention, detailed in Example 2 herein, PG-liposomes were administered to rats at 14 days, 13 days, and 1 day prior to testing for biochemical correlates of synaptic function, as further described below, with positive results. It is within routine testing to extrapolate such dosing regimens to other mammalian species.

The quantities of PG-carrying bodies to be administered will vary depending on the identity and characteristics of the patient. It is important that the effective amount of PG-bodies is non-toxic to the patient. The most effective amounts are unexpectedly small. When using intra-arterial, intravenous, subcutaneous or intramuscular administration of a liquid suspension of PG-carrying bodies, it is preferred to administer, for each dose, from about 0.1-50 ml of liquid, containing an amount of PG-carrying bodies generally equivalent to 10% -1000% of the number of leukocytes normally found in an equivalent volume of whole blood or the number of apoptotic bodies that can be generated from them. Generally, the number of PG-carrying bodies administered per delivery to a human patient is in the range from about 500 to about 2.5×10¹² (about 260 nanograms by weight), preferably from about 5,000 to about 500,000,000, more preferably from about 10,000 to about 10,000,000, and most preferably from about 200,000 to about 2,000,00

According to one feature of the invention, the number of such bodies administered to an Injection site for each administration is believed to be a more meaningful quantization than the number or weight of PG-carrying bodies per unit of patient body weight. Thus, it is contemplated that effective amounts or numbers of PG-carrying bodies for small animal use may not directly translate into effective amounts for larger mammals on a weight ratio basis.

It is contemplated that the PG-carrying bodies may be freeze-dried or lyophilized to a form which may be later resuspended for administration. This invention therefore also includes a kit of parts comprising lyophilized or freeze-dried PG- carrying bodies and a pharmaceutically acceptable carrier, such as physiological sterile saline, sterile water, pyrogen-free water, isotonic saline, and phosphate buffer solutions, as well as other non-toxic compatible substances used in pharmaceutical formulations. Such a kit may optionally provide injection or administration means for administering the composition to a subject.

IV. Utility

Compositions of the invention comprising phosphate-glycerol carrying bodies, and particularly phosphatidylglycerol (PG)-carrying bodies, may find use in treating or ameliorating the symptoms of ARML In aging subjects. Support for this feature of the invention is found, in part, in studies carried out in support of the invention, detailed In Example 2 and Example 3 described herein.

By way of description, but not limitation, studies carried out in support of the present invention have shown that when aged rats are given therapeutic dosages of PG-liposomes, brain levels of one or more biochemical markers of neuronal function improve, or trend toward improvement, where improvement is defined as moving in a direction of, or achieving a level not statistically significantly different from, levels of the biochemical marker exhibited by young animals.

More specifically, in studies carried out support of the present invention, hippocampal levels of certain age-elevated markers, specifically IFN-γ, pJNK, and IL-1β, decreased following a treatment regimen of PG-liposomes. On the other hand, the level of pERK, which was observed to decrease with age, was increased following PG-liposome treatment.

As described above, the rat hippocampus is thought to be a model for synaptic plasticity, which is also considered a surrogate for memory and learning. Thus, treatments that improve biochemical and/or electrophysiological correlates of synaptic function in the hippocampus are expected to improve memory and learning. The present invention has resulted in improvements in long term potentiation (LTP) in the hippocampus of aged animals, a form of synaptic plasticity. An indicator of LTP is the mean slope of the excitatory pos-synaptic potential (epsp) and its rate of decline to base levels after tetanic stimulation. Use of present invention causes a reduction in the rate, indicating improved memory features

Accordingly, it is contemplated that treatment of aging subjects with compositions of the invention will improve biochemical and electrophysiological components of the hippocampal region, particularly those involved in memory and cognition in humans. Such treatments are therefore contemplated to reduce or slow the progression of age-related memory loss (ARML) in mammalian subjects, including humans.

These results demonstrate a restoration of hippocampal function which has become impaired through age, to a level comparable to that in young animals, as a consequence of administration of PG liposomes. The results are, therefore, an indication for use of the treatment described herein to halt the progression of age-related memory impairment, and to restore memory function in mammalian patients experiencing a non dementia type decline in memory function to its previous, non-aged functioning level.

### EXAMPLES

The following examples are intended to illustrate methods for preparing therapeutic compositions of the present invention and exemplary treatment results. The examples are in no way intended to limit the scope of the invention.

### Example 1

### Preparation of Liposomes

A dry mixture (“Lipid Premix”) was prepared, consisting of semi-synthetic POPG (1-palmitoyl-2-oleoly-sn-glycero-3-phosphoglycerol sodium salt), 3 parts by mass, and POPC (1-palmitoyl-2-oleoyl-sn-glycero-3-phosphocholine), 1 part by mass.

The POPC ingredient was prepared from DPPC (dipalmitoyl-sn-glycero-3-phosphocholine) which was purified from soybean and enzymatically hydrolyzed with porcine pancreas phospholipase A2 (E.C. 3.1.1.4) to generate monopalmitoyl phosphatidylcholine (MPPC). The MPPC was acylated with oleic acid to generate POPC. The POPC was recovered and further purified by liquid phase chromatography to a purity of not less than 98%. The purified material was dried, dissolved in appropriate solvent (ethanol, t-butanol or chloroform), filtered through 0.22 micron filter and subsequently dried in a clean room.

The POPG ingredient was prepared from POPC. The POPC was dissolved in a suitable solvent (ethanol, t-butanol or chloroform) and incubated with excess glycerol in the presence of recombinant phospholipase D (E.C. 3.1.4.4). POPG was recovered and purified by liquid phase chromatography to a purity of not less than 98%. The material was dried, dissolved in appropriate solvent (ethanol, t-butanol or chloroform), filtered through 0.22 micron filter and subsequently dried in a clean room.

POPG and POPC were dissolved at a ratio of 3:1 by mass in t-butanol, followed by filtration (0.22 micron) and drying in a clean room, to form the Lipid Premix. These steps were performed for the Applicants by Lipoid GmbH, Frigensr.4.Ludwigshafen.

The Lipid Premix was hydrated with phosphate buffered saline (PBS, pH 7.0, sterilized by filtration through a 0.22 micron sterilizing filter). A suspension of multilamellar vesicles (MLVs) formed. The suspension was passed through polycarbonate filter (100 nm pore size) under pressure, generating unilamellar vesicles of about 100 nm in diameter. Vesicle size was verified, in-process, using a Quasi-Elastic Light Scattering (QELS) analysis. The suspension of unilamellar vesicles (liposomes) was immediately removed to a class 1,000 clean room, where it was redundantly filtered (0.22 micron) and filled into vials (1 mL per 2 mL amber vial) in a class 100 laminar flow hood. The vials were backfilled with nitrogen and sealed with butyl rubber stopper and aluminium crimp seals.

### Example 2

### Treatment with PG Liposomes

Male Wistar rats (BioResources Unit, Trinity College, Dublin, Ireland) of age 2-4 months (250-350 g; “young”) or 22-24 months (600-800 g; “aged”) were used in the experiments. They were assessed for hippocampal IFN-γ and IL-1β content, for JNK phosphorylation activity and for ERK phosphorylation activity, and for their ability to sustain long-term potentiation (LTP) in the hippocampus, with and without treatment according to the methods of the invention.

Aged animals were housed in pairs, and young animals in groups of 4-6, under 12 hour light schedule; ambient temperature was controlled between 22 and 23° C. and rats were maintained under veterinary supervision throughout the study. These experiments were performed under a license issued by the Department of Health (Ireland).

Aged male Wistar rats (22-24 months) and young male Wistar rats (2-4 months) were randomly assigned to four treatment groups; rats in two of these groups were injected with PG liposomes prepared as described in Example 1. Injections were made intramuscularly into the upper hind limb 14 days, 13 days, and 24 hours before treatment with anesthetic and subsequent assessment of the ability of rats to sustain LTP. Each injection for aged rats consisted of 300 microlitres of a 1.2×10⁷ particles/ml suspension in PBS (i.e., 3.6×10⁶ liposomes per injection). For young rats, each injection consisted of 150 microlitres of the same suspension. At corresponding times, the remaining two groups received three corresponding injections of saline. No local adverse effects were observed at any time.

On the day of the experiment, rats were anaesthetised by intraperitoneal injection of urethane (1.5 g per kilogram); the absence of a pedal reflex was considered to be an indicator of deep anaesthesia.

### Example 3

### Induction of LTP in vivo

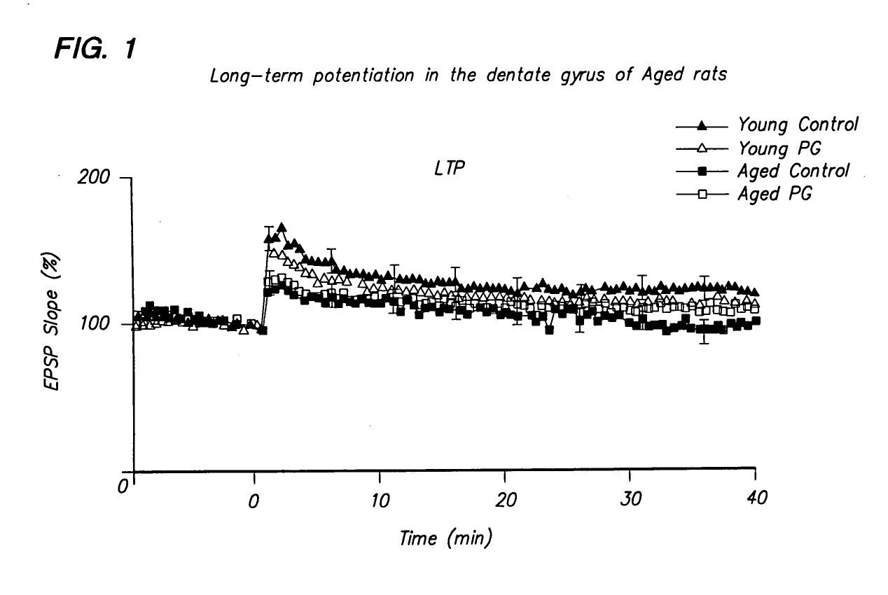

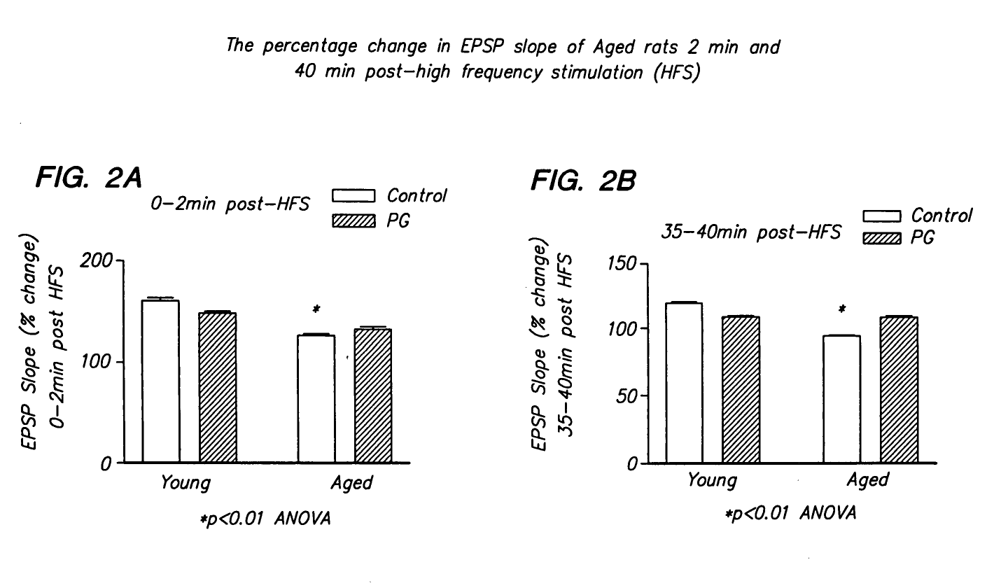

Analysis of LTP was conducted according to the method described by Vereker E, Campbell II: Roche E, McEntee E and Lynch M A, (2000) J: Biol. Chem 275: 26252-26258. Briefly, a bipolar stimulating electrode and a unipolar recording electrode were stereotaxically positioned in the perforant path (4.4 mm lateral to lambda) and dorsal cell body region of the dentate gyrus (2.5 mm lateral and 3.9 mm posterior to Bregma) respectively. Test shocks were delivered at 30 second intervals, and recorded for 10 minutes before and 40 minutes after tetanic stimulation (3 trains of stimuli; 250 Hz for 200 msec; 30 sec intertrain interval). The results are presented graphically as accompanying FIGS. 1 and 2.

FIG. 1 is a graph showing the difference in the excitatory post-synaptic potential (epsp) recorded in cell bodies of the granule cells. The data presented are means of seven to eight observations in each treatment group and are expressed as mean percentage change in epsp slope every 30 seconds, normalized with respect to the mean value in 5 minutes immediately prior to tetanic stimulation (time 0). FIG. 1 shows that LTP in perforant path-granule cell synapses was improved in aged rats with PG treatment (open squares) almost to the level of young rat controls (open triangles) and substantially better than that of aged rat controls (solid squares).

FIG. 2 graphically presents the same data somewhat differently, as the percentage change In epsp slope of the young and aged rats, with and without PG liposomes treatment, firstly at 0-2 minutes following high frequency stimulation and secondly at 35-40 minutes following high frequency stimulation (HFS). There is a significant (p<0.01 ANOVA) improvement in the treated aged rats over control aged rats, in both cases.

### Example 4

At the end of the experiment, rats were sacrificed by decapitation and the brain rapidly removed. The hippocampus was dissected free from the whole brain. Slices (350×350 micrometers) were prepared using a Mcllwain tissue chopper and stored in Krebs buffer containing calcium chloride (1.13 millimolar) and 10% DMSO at −80° C. until required for analysis, generally following methods described in Haan, E. A. and Bowen, D. M. (1981), J. Neurochem. 37, 243-246.

The concentrations of IL-1β and IFN-γ, were assessed in hippocampal homogenates, according to methods known in the art. In both cases, analysis was carried out by ELISA (R&D systems, U.K.). Hippocampal slices were thawed, and rinsed three times in ice cold Krebs solution and homogenized in ice cold Krebs solution. Protein concentrations in homogenates were equalized and triplicate aliquots (100 microliter) were used for ELISA. Biomarker-specific antibody-coated 96-well plates were incubated overnight at room temperature, washed several times with PBS containing 0.05% Tween 20, blocked for one hour at room temperature with blocking buffer (PBS, pH7.3; 5% sucrose; 1% BSA; 0.05% NaN₃), and incubated with standards or samples for two hours at room temperature. Wells were washed with PBS, incubated with secondary antibody for two hours at room temperature, washed again and incubated in horseradish peroxidase-conjugated streptavidin (1:200 dilution in PBS containing 1% BSA) for 20 minutes at room temperature. Substrate solution (1:1 mixture of hydrogen peroxide and tetramethylbenzidine) was added, incubation continued at room temperature in the dark for 30 minutes and reactions stopped using 1M sulfuric acid. Absorbance was read at 450 nm, the values were corrected for protein, and expressed as picagrams per milligram protein.

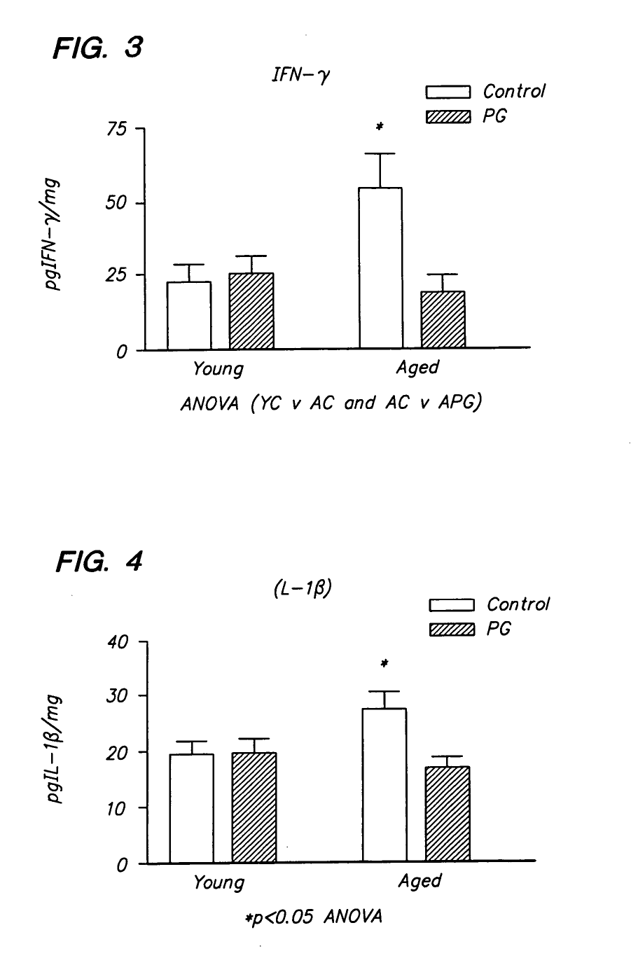

The results are presented as bar graphs, FIGS. 3 and 4. Inflammatory cytokine IFN-γ, substantially elevated in the hippocampus of untreated aged rats is shown on FIG. 3 to be reduced by the PG liposome treatment significantly down to the levels found for young rats. There was no significant difference between treated and untreated young rats. A similar result is shown in FIG. 4. The increased concentration of IL-1β found in the hippocampus of aged rats is shown to be significantly reduced to a level at or below that of young rats, by the PG liposome treatement. The treatment has no significant effect on Il-1β in the hippocampus of young rats.

### Example 5

### Assessment of JNK and ERK Activity

The phosphorylated forms of JNK (pJNK) and ERK (p-ERK) were assessed in homogenate obtained from the hippocampus of animals treated as described in Examples 2 and 4. Tissue samples prepared from the hippocampus were equalized for protein concentration, and aliquots (10 μl, 1 mg/ml) were added to sample buffer (5 μl; Tris-HCl, 0.5 mM, pH6.8; glycerol 10%; SDS, 10%; β-mercaptoethanol, 5%; bromophenol blue, 0.05% w/v), boiled for 5 minutes and loaded onto gels (12% SDS for JNK, 10% SDS for ERK). Proteins were separated by application of 30 mA constant current for 25-30 minutes transferred onto nitrocellulose strips (225 mA for 75 min) and immunoblotted with the appropriate antibody. To assess expression of p-JNK, nitrocellulose strips were incubated overnight at 4° C. in the presence of an antibody that specifically targets p-JNK (Santa Cruz, USA; diluted 1:200) in Tris buffered saline-Tween (TBS-T; 0.1% Tween-20) to which 0.1% BSA was added. Nitrocellulose strips were washed and incubated for 2 hours at room temperature with secondary antibody (peroxidase-linked anti-mouse IgG; 1:300 dilution Sigma UK), diluted in TBS-T containing 0.1% BSA. To assess expression of p-ERK, nitrocellulose strips were incubated overnight at 4° C. in the presence of an antibody that specifically targets p-ERK (Santa Cruz, USA, diluted 1:700) in phosphate buffered saline Tween and 6% dried milk, and incubated for 2 hours at room temperature with secondary antibody (anti-mouse 1gG; 1:1000 dilution) in PBS-Tween and 6% dried milk.

Protein complexes were visualized using Super Signal West Dura Extended Duration Substrate (Pierce, USA). Immunoblots were exposed to film for 1 to 10 s and processed using a Fuji x-ray processor. Protein bands were quantitated by densitometric analysis using Gel works software package (Gelworks ID, version 2.51; UVP Limited, UK), to provide a single value (in arbitrary units) representing the density of such blot.

FIG. 5 of the accompanying drawings shows that treatment of the aged animals with PG liposomes as described above results in a decrease in activation of JNK, a stress activated protein kinase that has been shown to trigger cell death in several cell types, including hippocampus.

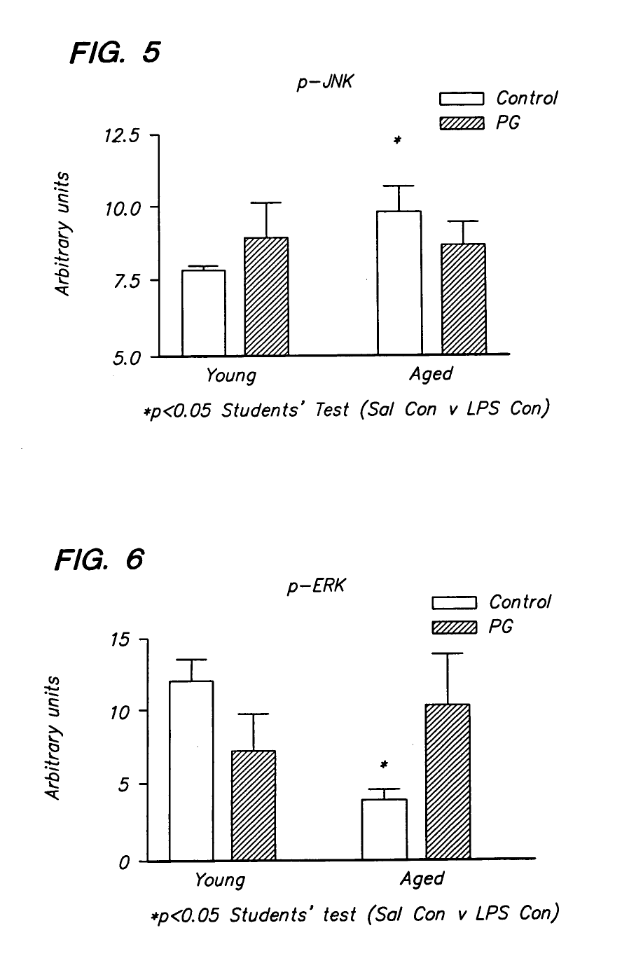

FIG. 6 of the accompanying drawings show that treatment of the aged animals with PG liposomes as described above results in an increase in activation of pERK, to close to the level found in untreated young animals.

What is claimed is:

1. A method for reducing symptoms associated with age related memory loss in a mammalian subject, comprising administering to the subject an effective amount of phosphatidylglycerol (PG)-carrying bodies.

2. The method of claim 1, wherein the mammalian subject is a human.

3. The method according to claim 2, wherein the PG-carrying bodies are liposomes constituted to the extent of 50% -100% by weight of phosphatidylglycerol.

4. The method according to claim 3, wherein the PG-carrying bodies have a diameter of from about 50 nanometers to about 1000 nanometers.

5. A method according to claim 4, wherein the PG-carrying bodies are administered in a unit dosage amount of from about 500 to about 5×10¹² bodies.

6. A method according to claim 5, wherein the PG-carrying bodies are administered intramuscularly.

7. A method of enhancing synaptic function in the brain of an aged mammalian subject, comprising administering to the subject, a therapeutically effective amount of phosphatidylglycerol (PG)-carrying bodies.

8. The method of claim 7, wherein the mammalian subject is a human.

9. The method according to claim 7, wherein the PG-carrying bodies are liposomes constituted to the extent of 50% - 100% by weight of phosphatidylglycerol.

10. A method according to claim 9, wherein the PG-carrying bodies have a diameter of from about 50 nanometers to about 1000 nanometers.

11. A method according to claim 10, wherein the PG-carrying bodies are administered in a unit dosage amount of from about 500 to about 5×10¹² bodies.

12. A method according to claim 11, wherein the PG-carrying bodies are administered intramuscularly.

13. A method according to claim 7, wherein said synaptic function is characterized by decreased hippocampal content of a biochemical marker selected from the group consisting of IFN-γ and IL-1β.

14. A method according to claim 7, wherein said synaptic function is characterized by increased hippocampal phosphorylation activity of the enzyme ERK.

15. A method according to claim 7, wherein said synaptic function is characterized by decreased hippocampal phosphorylation activity of the protein kinase JNK.
